(19) United States
(12) Patent Application Publication
(10) Pub. No.: US 2008/0020430 A1
(43) Pub. Date: Jan. 24, 2008

(54) Enzymatic Process for Preparing Aminoacyl Esters of Monosaccharides

(76) Inventors: Lohith Kenchaiah, Mysore (IN); Revanasiddappa Vijayakumar Giriyapura, Mysore (IN); Manohar Balaraman, Mysore (IN); Divakar Soundar, Mysore (IN)

Correspondence Address: LADAS & PARRY, 26 WEST 61ST STREET, NEW YORK, NY, 10023, US

(21) Appl. No.: 10/582,690

(22) PCT Filed: Dec. 31, 2003
(86) PCT No.: PCT/IN03/00466

§ 371 Date: May 15, 2007

(51) Int. Cl. C12P 19/26

(52) U.S. Cl. 435/84

(57) ABSTRACT

The present invention particularly relates to lipase mediated synthesis of aminoacyl esters of monosaccarides from unprotected amino acids and monosaccharides in non-polar solvents using lipases.

### FIELD OF THE INVENTION

The present invention provides an improved enzymatic process for the preparation of aminoacyl esters of monosaccharides. The present invention particularly relates to lipase mediated synthesis of aminoacyl esters of monosaccharides. The present invention further relates to preparation of aminoacyl esters of monosaccharides from unprotected amino acids and monosaccharides in non-polar solvents using lipases.

### BACKGROUND OF THE INVENTION

Aminoacyl esters of sugars are used as detergents, sweetening agents, as microcapsules in pharmaceutical preparations, in delivery of biological active agents, as antiviral nucleoside amino acid esters, as emulsions and as antibiotics. Regioselective acylation of carbohydrates is a challenging objective, quite difficult to achieve because of several hydroxyl groups in these molecules (Haines, A. H. Advances in Carbohydrate Chemistry and Biochemistry, 1976, 33, 11). Selective synthesis of these compounds using chemical reagents do not lead to products arising out of reactions at specific positions of the sugar molecules employed. Few regioselective transformations of carbohydrates have been carried out recently using enzymes (Wong, C. H. and Whitesides, G. M. Enzymes in organic synthesis, Elsevier Science, Oxford, 1994). Of these enzymatic synthesis of aminoacyl esters of sugars are very scanty.

Reference is made to Suzuki, Y., Shimizu, T., Takeda, H. And Kanda, K. Jpn Kokai Tokkyo Koho JP. 03216194 A2 24 Sep. 1991 wherein sugar esters of amino acids have been synthesised using enzymes from Neurospora sitophila and Rhodotorula lactosa.

Reference is made to Riva, S., Chopineau, J., Kieboom, A. P. G. and Klibanov, A. M. J. Amer. Chem. Soc., 110, 584-589, 1988, wherein 15 mmol of monosaccharides and 6 mmol of N-acetyl-L-phenylalanine chloroethyl ester dissolved in 30 mL of anhydrous dimethyl formamide containing 0.6 g subtilisin was shaken at 45° C. and 250 rpm for 16 h.

Reference is made to Oh-Jin Park, Gyo-Jong Jeon and Ji-Won Yang, Enzyme and Microbial Technology, 25, 455-462, 1999, wherein 1.03 g sucrose and 4.16 g of t-Boc-L-PheOTFE dissolved in 50 ml of pyridine at 45° C. in 250 ml round bottom flask containing 5 g of enzyme Optimase M-440 was shaken at 250 rpm for 8 days.

Reference is also made to Oh-Jin Park, Gyo-Jong Jeon and Ji-Won Yang, Biotech Letters, 18, 473-478, 1996 wherein a mixture of sugar and t-Boc-L-Phe-TFE sealed in a glass vial containg Optimase M-440 was shaken at 250 rpm at 45° C.

Reference is made to Yu Mitin, V., Kashparov, I. A., Kuhl, P. And Scheller, D. Bioorg. Khim., 25(4), 243-246, 1999, wherein sorbitol was esterified with N-benzyloxyvarbonyl alanine using papain.

Major drawbacks of the above mentioned enzymatic methods are:
1. The reactions were conducted at shake-flask levels using lesser quantities of the substrates and larger concentrations of the enzymes.
2. Several enzymes like Optimase M-440, subtilisin and from Neurospora sitophila and Rhodotorula lactosa were employed which are not readily available commercially.
3. Larger periods of incubation up to 8 days were employed.
4. The amino acids used were all derivatised to be protected at the amino positions. N-t-BOC and N-benzyloxycarbonyl groups were the protecting groups introduced.
5. In some cases the amino groups were also activated by derivatising at the carboxyl positions to undergo facile reaction. Mostly trifluoroethyl esters of amino acids were prepared before subjecting them to transesterification reaction with monosaccharides.

### OBJECTS OF THE INVENTION

The main object of the present invention is to provide an improved enzymatic process for preparing amino acyl esters of sugars which obviates the drawbacks as detailed above.

Another object of the present invention is to use underivatised amino acids without protecting amino groups or activating carboxyl groups.

Still another object of the present invention is to use underivatised monosaccharides as the sugar molecule Yet another object of the present invention is to provide a process for preparing amino acyl esters of sugars with large conversion.

It is another object of the invention to provide a process for preparing amino acyl esters of sugars with continuous removal of water thereby maintaining very low water activity essential for enzyme catalysed esterification reactions.

Yet another object of the present invention is to employ readily available commercial lipases like porcine pancreas lipase and Rhizomucor miehei lipase.

Yet another object of the present invention is to use lesser amounts of enzyme for achieving better conversions than what has been described in the above mentioned methods.

Still another object of the present invention is to use low boiling solvents in the temperature range 40° C.-80° C.

Yet another object of the present invention is to obtain aminoacyl esters of monosaccharides where a mixture of three monoesters, 6-O-aminoacyl, 3-O-aminoacyl and 2-O-aminoacyl esters are produced by the enzymatic reaction.

### SUMMARY OF THE INVENTION

Novelty of the present invention is that it uses underivatised amino acids where no amino group was protected and no carboxyl group was activated. An experimental set up was employed which facilitates use of larger concentrations of substrates and lesser amounts of enzymes. Commercially and readily available lipases can be employed. The procedure developed can be employed in the preparation of any aminoacyl sugar ester at even large-scale levels.

Accordingly, the present invention provides an improved enzymatic process for the preparation of an aminoacyl ester of a monosaccharide which comprises reacting an underivatised amino acid with a sugar in the presence of an enzyme and a non-polar solvent to produce an aminoacyl ester of a monosaccharide and recovering the product.

In one embodiment of the invention, the amino acid is without any N-protection and carboxyl activation.

In another embodiment of the invention, the amino acid is selected from the group consisting of glycine, L-alanine, L-valine, L-leucine, L-isoleucine, L-phenylalanine, L-tyrosine, L-histidine, L-tryptophan, L-lysine, L-aspartic acid, L-glutamic acid, L-arginine, L-serine, L-threonine and their corresponding D, L-mixtures.

In another embodiment of the invention, the underivatised sugar is a monosaccharide selected from the group consisting of D-glucose, D-fructose, D-galactose, D-mannose, D-arabinose, ribose and deoxyribose.

In another embodiment of the invention, the enzyme is a lipase selected from the group consisting of lipases obtained from porcine pancreas, Rhizomucor miehei, Candida cylindracea, Pseudomonas fluorescens and wheat germ.

In another embodiment of the invention, the solvent is a low boiling solvent having a boiling range 40° C.-80° C. and selected from the group consisting of dichloromethane, diisopropyl ether, chloroform, hexane, pentane, petroleum ether (60° C.-80° C. fraction), pyridine, dimethyl formamide, dimethyl sulfoxide, benzene and any mixture thereof

In another embodiment of the present invention, the reaction is carried out for a period in the range of 2-5 days.

In yet another embodiment of the invention, the reaction is carried out at a temperature in the range of 40° C.-80° C.

### DETAILED DESCRIPTION OF THE INVENTION

The present invention provides an improved enzymatic process for the preparation of an aminoacyl ester of a monosaccharide. The process comprises reacting an underivatised amino acid with a sugar in the presence of an enzyme and a non polar solvent preferably for a period of 2-5 days and at a temperature in the range 40° C.-80° C. The aminoacyl ester of a monosaccharide obtained are then recovered by conventional methods which are known in the art.

The amino acids employed are underivatised free ones without any N-protection and carboxyl activation. Examples of such amino acids include glycine, L-alanine, L-valine, L-leucine, L-isoleucine, L-phenylalanine, L-tyrosine, L-histidine, L-tryptophan, L-lysine, L-aspartic acid, L-glutamic acid, L-arginine, L-serine, L-threonine and their corresponding D, L-mixtures.

The underivatised sugar used for esterification is a monosaccharide including D-glucose, D-fructose, D-galactose, D-mannose, D-arabinose, ribose and deoxyribose. The enzyme used can be a commercially available lipase from porcine pancreas, Rhizomucor miehei, Candida cylindracea, Pseudomonas fluorescens and wheat germ.

The solvent employed is a low boiling solvent having a boiling range 40° C.-80° C. such as dichloromethane, diisopropyl ether, chloroform, hexane, pentane, petroleum ether (60° C.-80° C. fraction), pyridine, dimethyl formamide, dimethyl sulfoxide, benzene and any mixture thereof.

Porcine pancreas lipase, a Type II (Steapsin) was purchased from M/S Sigma Chemical. Co. Mo, USA. Lipozyme-IM20, a Rhizomucor meihei lipase, immobilized on a weak anion exchange resin, was obtained from Novo, Denmark. Esterification activities of porcine pancreas lipase and Lipozyme-IM20 were determined by an esterification procedure (Kiran, K. R., Hari Krishna, S., Suresh Babu, C. V., Karanth, N. G. and Divakar, S. Unpublished data, 1999). Activity assay measures butyric acid reacted after the enzyme was incubated with a mixture of butanol (0.32 M) and butyric acid (0.16 M) in n-heptane for a specified period of time. Protein was estimated by Lowry's method (Lowry, O. H., Roseborough, N. J., Farr, N. L. and Randall, R. J. J. Biol. Chem. 193, 265-275, 1951). Esterification activities of porcine pancreas lipase, and Lipozyme-IM20 were 0.06 and 0.46 μmoles/min/mg of the enzyme respectively.

Underivatised amino acid (0.001-0.01 mol) was taken in a flat bottomed two necked flask along with 50-500 ml of the solvent or a solvent mixture in presence of 0.1-2.0 g porcine pancreas lipase and refluxed for a period of 2-5 days in the temperature range of 40° C.-80° C. The reaction mixture was then added to 20-100 mL of water, stirred and filtered to remove the lipase. The filtrate was evaporated over a water bath to get a mixture of unreacted monosaccharides, unreacted amino acid and the aminoacyl esters of monosaccharides.

Percentage of esterification was determined by a back titration procedure whereby a known weight of the sample was treated with a known excess of 0.05 N KOH and the excess unreacted KOH was titrated against 0.1 N oxalic acid. From the titre value corresponding to the free acid the amount of free acid was determined. From the known weight of the amino acid employed the extent of ester formed was determined. The aminoacyl ester of monosaccharides was characterized by recording one-dimensional ¹H and ¹³C NMR and two-dimensional NMR spectra on a Brüker DRX-400 and 500 NMR instruments operating at 20° C. Samples were dissolved in DMSO-d₆ and D₂O and the signals were referenced to DSS.

The reaction mixture on analysis showed the formation of three monoesters and about 5% of peptides.

NMR data for L-Phenylalanine glucose esters:
6-O-Ester: ¹H δₚₚₘ: βCH₂ₐ-3.26; βCH_{2b}-2.94; Aromatic H_{2,6}-7.23; H_{3,5}-7.29; H₄-7.24; H₁-4.93; H₄-3.47; H₅-3.64; H₆a-3.72; H₆b-3.45; ¹³C δₚₚₘ: βC-36.2; Aromatic-C₁-137.1; C₂-127.6; C₃-130.0; C₄-127.8; C₅-130.1; C₆-127.6. C₁α-101.8; C₄α-71.0; C₅α-78.7; C₆α-64.4; C₀-170.7.
3-O-Ester: ¹H δₚₚₘ: βCH₂ₐ-2.93; βCH_{2b-2.80}; αCH-3.57; H₃-3.76; H₃-3.88. ¹³C δₚₚₘ: βC-36.3: αC-53.6; Aromatic-C₁-137.2; C₁α-99.9; C₃α-82.5; C₃β-83.5; Cₒ-170.9.
2-O-Ester: ¹H δₚₚₘ: βCH₂ₐ-2.95; βCH_{2b}-2.82; αCH-3.13; H₂-3.45. ¹³C δₚₚₘ: αC-52.6; C₁α-95.8; C₂α-77.0; C₂β-75.1; Cₒ-171.4.

NMR data for L-Leucine monosaccharides esters:
6-O-Ester: ¹H δₚₚₘ: αCH-3.05; γ-CH-1.65; δ-CH₃-0.55; ε-CH₃-0.50; H₃-3.8; H₃-3.95; H₄-3.74; H₆a-3.7; H₆b-3.45. ¹³C δₚₚₘ: αC-53.0; γ-C-20.9; δ-C-11.5; ε-C-14.0; Co-172.5; C₃α-83.0; C₃β-84.0; C₄α-70.0; C₆α-64.9.
3-O-Ester ¹H δₚₚₘ: γCH-2.69

The following examples are given by way of illustration and therefore should not be construed to limit the scope of the present invention.

### EXAMPLE 1

A 0.025 mol of D-glucose and 0.025 mol of free L-phenylalanine was taken in 100 ml dimethyl formamide: dichloromethane mixture (1:9) in a two-necked round bottomed flask fitted with a Soxhelet apparatus filled with molecular sieves. The reaction mixture was treated with 0.09 g of Lipozyme IM-20, a Rhizomucor miehei lipase immobilized on weak anion exchange resin and refluxed at 40° C. for a period of 72 h. Continuous removal of water was achieved by condensing the solvent vapours into molecular sieves before they were drained into the flask. After the reaction, the reaction mixture was then added to 50 mL of water, stirred and filtered to remove the lipase. The filtrate was evaporated over a water bath to the get a mixture of unreacted monosaccharides, unreacted amino acid and the L-phenylalanyl glucose esters. The reaction by the back titration method gave a: conversion yield of 27.0% and by HPLC 36.5%.

### EXAMPLE 2

0.001 mol of glucose and 0.001 mol of free L-phenylalanine was taken in 100 ml dimethyl formamide: dichloromethane mixture (1:9) in a two-necked round bottomed flask fitted with a Soxhelet apparatus filled with molecular sieves. The reaction mixture was treated with 0.036 g of porcine pancreas lipase and refluxed at 40° C. for a period of 72 h. Continuous removal of water was achieved by condensing the solvent vapours into molecular sieves before they were drained into the flask. After the reaction, the reaction mixture was then added to 50 mL of water, stirred and filtered to remove the lipase. The filtrate was evaporated over a water bath to the get a mixture of unreacted monosaccharides, unreacted amino acid and the L-phenylalanyl glucose esters. The reaction by the back titration method gave a conversion yield of 36.5% and by HPLC 56.5%

### EXAMPLE 3

0.001 mol of glucose and 0.003 mol of free L-phenylalanine was taken in 100 ml dimethyl formamide: dichloromethane mixture (1:9) in a two-necked round bottomed flask fitted with a Soxhelet apparatus filled with molecular sieves. The reaction mixture was treated with 0.036 g, of porcine pancreas lipase and refluxed at 40° C. for a period of 72 h. Continuous removal of water was achieved by condensing the solvent vapours into molecular sieves before they were drained into the flask. After the reaction, the reaction mixture was then added to 50 mL of water, stirred and filtered to remove the lipase. The filtrate was evaporated over a water bath to the get a mixture of unreacted monosaccharides, unreacted amino acid and the L-phenylalanyl glucose esters. The reaction by the back titration method gave a conversion yield of 57.0% and by HPLC 52.0%.

### EXAMPLE 4

0.001 mol of glucose and 0.001 mol of free L-leucine was taken in 100 ml dimethyl formamide: dichloromethane mixture (1:9) in a two-necked round bottomed flask fitted with a Soxhelet apparatus filled with molecular sieves. The reaction mixture was treated with 0.055 g of Lipozyme IM-20, a Rhizomucor miehei lipase immobilized on weak anion exchange resin and refluxed at 40° C. for a period of 72 h. Continuous removal of water was achieved by condensing the solvent vapours into molecular sieves before they were drained into the flask. After the reaction, the reaction mixture was then added to 50 mL of water, stirred and filtered to remove the lipase. The filtrate was evaporated over a water bath to the get a mixture of unreacted monosaccharides, unreacted amino acid and the L-leucyl glucose esters. The reaction by the back titration method gave a conversion yield of 75.3% and by HPLC 54.7%.

### EXAMPLE 5

0.001 mol of glucose and 0.001 mol of free L-leucine was taken in 100 ml dimethyl formamide: dichloromethane mixture (1:9) in a two-necked round bottomed flask fitted with a Soxhelet apparatus filled with molecular sieves. The reaction mixture was treated with 0.072 g of porcine pancreas and refluxed at 40° C. for a period of 72 h. Continuous removal of water was achieved by condensing the solvent vapours into molecular sieves before they were drained into the flask. After the reaction, the reaction mixture was then added to 50 mL of water, stirred and filtered to remove the lipase. The filtrate was evaporated over a water bath to the get a mixture of unreacted monosaccharides, unreacted amino acid and the L-leucyl glucose esters. The reaction by the back titration method gave a conversion yield of 43.8% and by HPLC 68.0%.

### EXAMPLE 6

0.001 mol of glucose and 0.005 mol of free L-leucine was taken in 100 ml dimethyl formamide: dichloromethane mixture (1:9) in a two-necked round bottomed flask fitted with a Soxhelet apparatus filled with molecular sieves. The reaction mixture was treated with 0.054 g of Lipozyme IM-20, a Rhizomucor miehei lipase immobilized on weak anion exachange resin and refluxed at 40° C. for a period of 72 h. Continuous removal of water was achieved by condensing the solvent vapours into molecular sieves before they were drained into the flask. After the reaction, the reaction mixture was then added to 50 mL of water, stirred and filtered to remove the lipase. The filtrate was evaporated over a water bath to the get a mixture of unreacted monosaccharides, unreacted amino acid and the L-leucyl glucose esters. The reaction by the back titration method gave a conversion yield of 84.4%.

The main advantages of the invention are:
1. Employment of underivatised amino acids. Even derivatised amino acids can be employed.
2. Larger conversions were achieved by carrying out reactions in a specially designed experimental setup.
3. An experimental set up developed enabled continuous removal of water of reaction which otherwise resulted in reducing the extent of esterification due to hydrolysis.
4. Use of a readily available commercial lipase like porcine pancreas lipase Rhizomicor miehei lipase.
5. Employment of lesser amount of the enzyme for achieving better conversions.
6. This method does not involve any derivatisation of Monosaccharides.
7. Employment of low boiling solvents in the temperature range 40° C.-80° C.
8. This method can be employed for obtaining conversions at even large-scale levels.

We claim:

1. A enzymatic process for the preparation of an aminoacyl ester of a monosaccharide which comprises reacting an underivatised amino acid with a sugar in the presence of an enzyme and a non-polar solvent to produce an aminoacyl ester of a monosaccharide and recovering the product.

2. A process as claimed in claim 1 wherein the amino acid is without any N-protection and carboxyl activation.

3. A process as claimed in claim 1 wherein the amino acid is selected from the group consisting of glycine, L-alanine, L-valine, L-leucine, L-isoleucine, L-phenylalanine, L-tyrosine, L-histidine, L-tryptophan, L-lysine, L-aspartic acid, L-glutamic acid, L-arginine, L-serine, L-threonine and their corresponding D, L-mixtures.

4. A process as claimed in claim 1 wherein the underivatised sugar is a monosaccharide selected from the group consisting of D-glucose, D-fructose, D-galactose, D-mannose, D-arabinose, ribose and deoxyribose.

5. A process as claimed in claim 1 wherein the enzyme is a lipase selected from the group consisting of lipases obtained from porcine pancreas, Rhizomucor miehei, Candida cylindracea, Pseudomonas fluorescens and wheat germ.

6. A process as claimed in claim 1 wherein the solvent is a low boiling solvent having a boiling range 40° C.-80° C. and selected from the group consisting of dichloromethane, diisopropyl ether, chloroform, hexane, pentane, petroleum ether (60° C.-80° C. fraction), pyridine, dimethyl formamide, dimethyl sulfoxide, benzene and any mixture thereof.

7. A process as claimed in claim 1 wherein the the reaction is carried out for a period in the range of 2-5 days.

8. A process as claimed in claim 1 wherein the reaction is carried out at a temperature in the range of 40° C.-80° C.

9. A process as claimed in claim 1 wherein the product is separated by filtration.
